(19) United States
(12) Patent Application Publication
(10) Pub. No.: US 2014/0187648 A1
(43) Pub. Date: Jul. 3, 2014

(54) Identifying Subjects in Need of Treatment

(71) Applicant: HOWARD FOUNDATION HOLDINGS LTD., Great Shelford (GB)

(72) Inventors: Alan N. Howard, Great Shelford (GB); John Nolan, Williams Town (IE); Stephen Beatty, Butlertown (IE)

(73) Assignee: HOWARD FOUNDATION HOLDINGS LTD., Great Shelford (GB)

(21) Appl. No.: 14/146,042

(22) Filed: Jan. 2, 2014

Related U.S. Application Data
(63) Continuation-in-part of application No. 13/752,723, filed on Jan. 29, 2013.

(30) Foreign Application Priority Data
Jan. 3, 2013 (GB) 1300055.9

(51) Int. Cl. A61K 31/047; A61B 5/00

(52) U.S. Cl. CPC A61K 31/047; A61B 5/4088
USPC 514/729; 600/300

(57) ABSTRACT

A method of identifying a human subject, more likely than a subject selected at random from the general population, to have one or more of the following: (i) a low macular pigment concentration in the eye or eyes; (ii) a low visual performance, or (iii) an atypical “central dip” macular pigment distribution; the method comprising the steps of: measuring at least one cognitive function of the subject; comparing the measured cognitive function with a pre-determined threshold; and, if the measured cognitive function is below the threshold, declaring the subject as being more likely to have one or more of low macular pigment concentration in the eye or eyes, low visual performance or an atypical ‘central dip’ macular pigment profile.

### FIELD OF THE INVENTION

This invention relates to a method of identifying a human subject in need of dietary supplementation with meso-zeaxanthin.

### BACKGROUND OF THE INVENTION

The central retina, known as the macula, is responsible for colour and fine-detail vision. A pigment, composed of the two dietary carotenoids, lutein (L) and zeaxanthin (Z), and a typically minimal-dietary carotenoid meso-zeaxanthin (MZ), accumulates at the macula where it is known as macular pigment (MP). MP is a blue light filter and a powerful antioxidant, and is therefore believed to protect against age-related macular degeneration (AMD), which is now the most common cause of blind registration in the western world.

MZ-containing compositions have been disclosed as useful in the treatment of age-related macular degeneration (AMD), see for example U.S. Pat. No. 6,329,432. Supplements containing each of L, Z and MZ are known, and sold for the intended purpose of treating and/or preventing eye disorders such as AMD. One example of such a supplement is sold under the trade mark MacuShield®, and contains the three MP carotenoids L, Z and MZ in the amounts of 10 mg, 2 mg and 10 mg respectively, per dose.

WO 03/063848 discloses the use of a compound, such as lutein, zeaxanthin, mesozeaxanthin or mixtures thereof, for the manufacture of a composition for improving visual performance of a subject in conditions of darkness. The document is however rather unusual in that it does not contain any experimental evidence or data to support the alleged use.

EP 1 920 711 discloses a method of assessing visual performance which, in effect, involves measuring or determining the amount of macular pigment (such as lutein, zeaxanthin or mesozeaxanthin) present in the subject's eye (i.e. measuring macular pigment optical density, MPOD). If the level of MPOD is low, the document suggests administering a composition comprising lutein and/or zeaxanthin, which is purported to lead to an improvement in visual performance. However, the document does not disclose any actual experimental data to show that improving the level of macular pigment can produce an improvement in visual performance. The person skilled in the art would therefore treat the disclosure of the document with some caution and could not derive any expectation of success therefrom.

Our unpublished patent application (PCT/GB2012/051567) presents experimental data which demonstrates that dietary supplementation with a composition comprising macular carotenoid can improve visual performance in human subjects.

In particular we have discovered that consumption of a dietary supplement containing lutein alone has little effect in the MP of subjects who exhibit an abnormally low concentration of MP in the central portion of the retina. In contrast, consumption of a dietary supplement comprising MZ alone can return MP levels in the central portion of the retina substantially to normal, but has little effect on MP levels outside the central portion. Consumption of a combined supplement, containing relatively high amounts of MZ, but also Z and L, can not only normalise MP levels in the central region of the retina, but also augment MP levels outside the central region of the retina.

For present purposes, the ‘central region’ of the retina means that central portion of the retina which has an eccentricity of 0.25° or less, as determined by optical coherence tomography (OCT) and/or fundus photography.

It has been found in one study that about 12% of the normal population in Ireland had an abnormal profile of macular pigmentation (without showing any clinical signs of age-related macular degeneration i.e. were free of AMD). The normal profile has a peak concentration of macular pigments in the central portion of the macula, with the concentration of pigment declining sharply with increasing eccentricity. In the subjects with an abnormal macular pigment profile, the peak pigment concentration was found slightly outside the central portion. For this reason the atypical profile may be referred to as a “central dip” (i.e. an MPOD at 0.25° retinal eccentricity less than or equal to that at 0.5° retinal eccentricity). PCT/GB2012/051567 (unpublished at date of filing of the present application) presents the results of experimental trials which demonstrate that the macular pigment distribution in subjects with such an atypical “central dip” profile can be normalised by the consumption of a dietary supplement containing macular carotenoids. Normal subjects who have an atypical spatial profile (central dip) can be detected by using a densitometer (see Nolan et al., “Macular carotenoid supplementation in subjects with atypical spatial profiles” Exp. Eye Res. 101 (2012) 9-15). However Alzheimer's disease subjects, because of their low cognitive ability, cannot with any reliance be diagnosed as having a central dip by the above method because it requires them to respond verbally to the presence or absence of a flicker of light. We have shown that a central dip in these patients can be detected using the dual-wavelength fundus auto fluorescence (AF) technique (Spectralis Heidelberg Engineering technology). The latter is very expensive and currently only available in specialised research laboratories. Although Alzheimer patients can easily be tested for contrast sensitivity, the equipment and methodology is likewise restricted to research establishments. Both normal and Alzheimer Disease subjects would gain exceptional benefit from dietary supplementation with macular cartotenoid containing mesozeaxanthin. It is proposed because of the abovementioned factors that the benefits of this invention would apply especially to Alzheimer's disease subjects

### SUMMARY OF THE INVENTION

The present inventors have surprisingly found that macular pigment concentration and visual performance (especially contrast sensitivity) are frequently depressed in subject with Alzheimer's disease compared with aged matched controls; and the prevalence of an atypical “central dip” macular pigment profile in subjects with Alzheimer's disease is much higher (about 50%) than would be expected in the general population (about 12%).

This is significant because suspected cases of Alzheimer's disease can be identified readily by simply testing selected aspects of the subject's cognitive function, without requiring access to expensive apparatus. Whilst it is true that, at present, a definitive clinical diagnosis of Alzheimer's disease requires more extensive investigation, such is not necessary to identify suspected cases.

Accordingly, simply by measuring cognitive function of the subject, and comparing the result with some pre-determined threshold, one can identify a subject suspected of having Alzheimer's disease and therefore being more likely, than a subject selected at random from the general population, to benefit from dietary supplementation with a macular carotenoid composition.

Thus, in a first aspect the invention provides a method of identifying a human subject, more likely than a subject selected at random from the general population, to have one or more of the following: (i) a low macular pigment concentration in the eye or eyes; (ii) a low visual performance; or (iii) an atypical ‘central dip’ macular pigment distribution; the method comprising the steps of: measuring at least one cognitive function of the subject; comparing the measured cognitive function with a pre-determined threshold; and, if the measured cognitive function is below the threshold, declaring the subject as being more likely to have a low macular pigment concentration and/or low visual performance (especially contrast sensitivity) and/or an atypical ‘central dip’ macular pigment profile.

The pre-determined threshold may be a threshold derived from a mean value pertaining to the general population (optionally corrected to compensate for individual factors, such as age, education, etc.). Alternatively, the threshold may be one derived from one or more measurements of cognitive function in the same individual performed previously, which will show if the individual's cognitive function is declining and, if so, at a rate that might be greater than expected for a normal healthy individual.

The step of measuring cognitive function may therefore comprise an absolute measurement and/or a relative measurement. The particular cognitive function or functions selected to be measured, and the most appropriate techniques chosen to perform the measurement, will be apparent to those skilled in the art. In general terms, suitable cognitive function measurement techniques include, but are not limited to, the following: the standard MMSE (mini-mental state examination); FAS (phonemic fluency score) and Animal fluency score (semantic fluency score).

These tests are common general knowledge for the person skilled in the art and widely used by clinicians to help diagnose dementia and to assess its severity and/or progression. Standard lists of questions or tasks to be performed by a subject during the MMSE are widely available on the internet. The phonemic fluency score (FAS) and the animal fluency score may be assessed using well-known techniques. The ‘FAS’ test involves a test subject being asked to list, in a 60-second period, as many words as possible starting with the letter F, A or S (each letter tested separately), (see e.g. Spreen & Strauss “A compendium of neurophysiological tests: Administration, Norms and Commentary” 2^{nd} Edition Oxford University Press; New York 1998). The “animal fluency” test involves a test subject being asked to name, in a 60 second period, as many animals as possible; (see, for example, Sager et al, “Screening for dementia in Community-based memory clinics” Wisconsin Medical Journal 2006, 105(7) 25-29).

Cognitive function may also be measured and assessed using a technology platform called CANTAB® (Cambridge Neuropsychological Test Automated Battery [www.cambridgecognition.com]). If desired two or more of the foregoing tests may be used on a subject. For example, both a phonemic fluency test and an animal fluency test may be used, as both are very simple and quick to perform. If desired, a subject may be tested using three or more of the foregoing tests.

For present purposes, a subject may be considered to exhibit a low macular pigment concentration in the eye or eyes, if macular pigment optical density (MPOD), at 0.25° eccentricity, in at least one of the subject's eyes, is 0.48 or less, preferably 0.46 or less, more preferably 0.44 or less, as measured by the method described by Nolan et al (2012 Experimental Eye Research 101, 9-15) using a macular densitometer.

Also for present purposes, a subject is considered to exhibit a low visual performance if, as assessed by letter contrast sensitivity according to the method disclosed by Loughman et al (2010 Vision Res. 50, 1249-1256) and as described herein, the subject has a letter contrast sensitivity for any particular spatial frequency, which is at least one standard deviation (1 S.D.) lower, preferably at least 1.5 standard deviations, more preferably at least 2 standard deviations lower, than the mean for a representative sample of the normal population of human subjects (e.g. without underlying ocular disease and otherwise in good health, aged 18-70, etc.).

For present purposes, a subject has a ‘central dip’ macular pigment distribution if they exhibit an MPOD at 0.25° eccentricity less than or equal to that at 0.5° retinal eccentricity, as defined previously.

In a second aspect, the invention provides a method of improving the visual performance of a human subject without the need to test the macular pigment concentration or the visual performance in the eye or eyes of the subject, the method comprising the steps of: identifying a subject likely to have low macular pigment concentration, low visual performance and an atypical ‘central dip’ macular pigment distribution in accordance with the first aspect of the invention defined above; and administering an amount of a macular pigment-containing composition sufficient to improve the visual performance of the subject.

Preferably the macular pigment-containing composition is administered orally, and most preferably as a dietary supplement. Suitable dietary supplements are commercially available and include, for example, Macushield®, available from several different retailers.

Preferably the macular pigment-containing composition comprises mesozeaxanthin, which is present in the typical human diet in very small quantities only and is thought to accumulate in the macula as a result of conversion from lutein.

In one embodiment mesozeaxanthin is the sole, or predominant, macular pigment present in the composition. In one embodiment mesozeaxanthin is the predominant molecular pigment present. In some embodiments, lutein and/or zeaxanthin may additionally be present.

In a further aspect the invention provides for use of a macular pigment-containing composition to improve the visual performance of a subject suffering from Alzheimer's disease. Generally, the subject will have low macular pigment concentration and low visual performance and an atypical ‘central dip’ macular pigment profile, and may preferably be selected for treatment on the basis of an assessment of cognitive function (e.g. a putative diagnosis of Alzheimer's disease) alone. Optionally, selection for treatment may also be based on an assessment of visual performance (especially letter contrast sensitivity) and/or by direct assessment of macular pigment concentration and/or distribution.

The invention also provides a method of improving the visual performance of a subject, the method comprising the step of administering an effective amount of a macular pigment containing composition to an individual in need of such treatment, for a period sufficient to improve the individual's visual performance.

### Thresholds for Impaired Cognition

The following are different measures of cognitive function known to those skilled in the art. They provide a set of thresholds appropriate for the working of this invention.
1. Mini-Mental State Examination (MMSE)—this is used to screen for cognitive impairment in clinical practice). The MMSE is a 30-point scale with 24 being the cut-off for screening for dementia. The cut-off is variable depending upon a number of factors including education. (See Serge Gauthier & Clive Ballard, 2009 Management of Dementia (2^{nd} edition), Informa Healthcare USA Inc.

In Example 2 described below, the average MMSE was 18±4 in patients with AD and was 29±1 in age-matched controls (p<0.01).
2. Phonemic fluency score (referred to as “FAS”, since the test uses the letters F, A and S) The threshold here is difficult to confirm as it is dependent on age and education. The normative data are presented in Table 1 below. Anything less than these mean values is indicative of AD, for present purposes.

In Example 2 described below the average FAS score was 19±12 in patients with AD and was 31±11 in age-matched controls (p<0.038).

TABLE 1
Norms for FAS scores Stratified for age and years of education are presented below.

| | Age 16-59 Years | | | Age 60-79 Years | | | Age 80-95 Years | | |
|---|---|---|---|---|---|---|---|---|---|
| | Education (Years) | | | Education (Years) | | | Education (Years) | | |
| Percentile | 0-8 | 9-12 | 13-21 | 0-8 | 9-12 | 13-21 | 0-8 | 9-12 | 13-21 |
| Score | (n = 12) | (n = 268) | (n = 242) | (n = 76) | (n = 292) | (n = 185) | (n = 75) | (n = 102) | (n = 46) |
| 90 | 48 | 56 | 61 | 39 | 54 | 59 | 33 | 42 | 56 |
| 80 | 45 | 50 | 55 | 36 | 47 | 53 | 29 | 38 | 47 |
| 70 | 42 | 47 | 51 | 31 | 43 | 49 | 26 | 34 | 43 |
| 60 | 39 | 43 | 49 | 27 | 39 | 45 | 24 | 31 | 39 |
| 50 | 36 | 40 | 45 | 25 | 35 | 41 | 22 | 29 | 36 |
| 40 | 35 | 38 | 42 | 22 | 32 | 38 | 21 | 27 | 33 |
| 30 | 34 | 35 | 38 | 20 | 28 | 36 | 19 | 24 | 30 |
| 20 | 30 | 32 | 35 | 17 | 24 | 34 | 17 | 22 | 28 |
| 10 | 27 | 28 | 30 | 13 | 21 | 27 | 13 | 18 | 23 |
| M | 38.5 | 40.5 | 44.7 | 25.3 | 35.6 | 42.0 | 22.4 | 29.8 | 37.0 |
| (SD) | (12.0) | (10.7) | (11.2) | (11.1) | (12.5) | (12.1) | (8.2) | (11.4) | (11.2) |

Note.
M = mean;
SD = standard deviation.
(Source: Tombaugh, T. N., Kozak, J., & Rees, L. 1999; “Normative data stratified by age and education for two measures of verbal fluency: FAS and animal naming” Arch. Clin. Neuropsychol. 14(2), 167-177).

3. Animal Fluency Score

The threshold here is difficult to confirm as it is dependent on age and education. The normative data are presented in Table 2 below. Anything less than these mean values is indicative of AD, for present purposes.

In Example 2 described below, the average Animal Fluency score was 7±3 in patients with AD and was 16±3 in age-matched controls (p<0.038).

TABLE 2
Norms for Animal Fluency Scores Stratified for age and years of education

| | Age 16-59 Years | | | Age 60-79 Years | | | Age 80-95 Years | | |
|---|---|---|---|---|---|---|---|---|---|
| | Education (Years) | | | Education (Years) | | | Education (Years) | | |
| Percentile | 0-8 | 9-12 | 13-21 | 0-8 | 9-12 | 13-21 | 0-8 | 9-12 | 13-21 |
| Score | (n = 4) | (n = 109) | (n = 78) | (n = 61) | (n = 165) | (n = 94) | (n = 75) | (n = 103) | (n = 46) |
| 90 | | 26 | 30 | 20 | 22 | 25 | 18 | 19 | 24 |
| 75 | | 23 | 25 | 17 | 19 | 22 | 16 | 17 | 20 |
| 50 | | 20 | 23 | 14 | 17 | 19 | 13 | 14 | 16 |
| 25 | | 17 | 18 | 12 | 14 | 16 | 11 | 12 | 14 |
| 10 | | 15 | 16 | 11 | 12 | 13 | 9 | 11 | 12 |
| M | | 19.8 | 21.9 | 14.4 | 16.4 | 18.2 | 13.1 | 13.9 | 16.3 |
| (SD) | | (4.2) | (5.4) | (3.4) | (4.3) | (4.2) | (3.8) | (3.4) | (4.3) |

Note.
M = mean;
SD = standard deviation.
Source: Tombaugh, T. N. et al. 1999

4. Verbal Recognition Memory (VRM) test assesses immediate and delayed memory of verbal information under free recall and forced choice recognition. No normative data available.

In Example 2 described below, control subjects scored 5 to 9 (out of 12) on the Free Recall VRM phase, while AD patients only scored 1 to 2. On the Immediate Recognition VRM phase, control subjects scored 21 to 24 (out of 24), while AD patients scored only 15 to 22.
5. Paired Associates Learning (PAL)

A normative database is available as part of the CANTAB device (see Example 2). The CANTAB eclipse application calculates standardised scores from subjects' raw test scores using this normative database.

In Example 2 described below, control subjects scored 0-30 (mean 14) and AD patients scored 30 on the PAL test (total errors, adjusted). Of note, for this measure, lower scores indicate better cognitive function.

The CANTAB computerized cognitive testing is not currently being used in clinical practice to diagnose impaired cognition or Alzheimer's disease. Therefore, there are no established thresholds for cognitive impairment.

Considering the data in Example 2 cross-sectional study, we can speculate that subjects who score less than 5 on the Free Recall VRM test, less than 21 on the Immediate Recognition VRM test, and more than 29 on the PAL test (total errors, adjusted) are likely to have memory deficits.

### Visual Performance

Visual performance is a parameter, not an abnormality or a disease in itself. Thus there is a range of values in normal human subjects without necessarily the presence of any underlying retinal or macular disease, although poor visual performance may sometimes be a result and/or indication of some underlying pathological condition.

There are many different measures of “visual performance” known to those skilled in the art.

For present purposes, improving “visual performance” means producing a detectable improvement in one or more of the following in the subject: contrast sensitivity; visual acuity, preferably best corrected visual acuity; glare disability; discomfort glare; ocular straylight; photostress recovery; and S-cone sensitivity. Preferably the improvement in visual performance created by consumption of the composition of the invention comprises an improvement in one or more of: contrast sensitivity, best corrected visual activity, or glare disability. Contrast sensitivity is the preferred parameter, especially letter contrast sensitivity. A suitable method of measuring contrast sensitivity is that disclosed by Loughman et al (2010 Vision Res. 50, 1249-1256) as summarised and discussed herein below.

Preferably consumption of the macular pigment-containing composition will produce a detectable improvement in one or more of the parameters of visual performance, more preferably in two or more, and most preferably a detectable improvement in three or more of the aforementioned visual performance parameters.

The various parameters of visual performance listed above are described in more detail below.

### (i) Contrast Sensitivity

Contrast is the difference in visual properties that make an object (or its representation in an image) distinguishable from other objects and the background. In visual perception of the real world, contrast is determined by the difference in the colour and brightness of the object and other objects within the same field of view. Contrast Sensitivity is a measure of a subject's sensitivity to changes in contrast; it is a measure of how much contrast is required to accurately detect a target as distinct from its background.

By altering the size (spatial frequency) of a target, and the luminance of the background, it is possible to test Contrast Sensitivity, which is very much reflective of real-world vision, where the most important determinants of vision are contrast, size and luminance. Contrast Sensitivity function can be assessed using the Functional Acuity Contrast Test (FACT), which is designed to test contrast sensitivity at varying spatial frequency settings, as disclosed by Loughman et al., 2010 Vision Res. 50, 1249-1256). Letter Contrast Sensitivity may be measured using the commercially available “Thomson Chart”.

### (ii) Visual Acuity

Visual acuity is a simple and intuitive way of assessing visual performance It is a useful measure of vision because it relates directly to the need for spectacles (i.e. if an individual is long or short sighted, the introduction of spectacle lenses typically creates a predictable improvement in visual acuity). Also, it tends to be adversely affected by ocular disease and therefore abnormal visual acuity can be a sign of developing abnormality.

Despite its widespread use and popularity, it is not the best technique for the assessment of vision because (a) it tends not to relate well with vision in conditions different to the brightly lit, high contrast test environment, and (b) it only evaluates performance at the high spatial frequency (i.e. small letter size) end of the spectrum.

Typically best corrected visual acuity (“BCVA”) is assessed using a high contrast (close to 100%, i.e. black letters on a white background) letter chart, after the subject's vision has been corrected with corrective lenses to the best level possible. The subject's task is to read the smallest possible letter size they can recognise. The visual performance is quantified using a standard notation (e.g. Snellen notation; where 20/20 or 6/6 vision is accepted as normal human vision). Improvements in BCVA imply a benefit in visual acuity in general.

(iii) Glare Disability

Glare disability is a term used to describe the degradation of visual performance typically caused by loss of retinal image contrast. Glare disability if often caused, for example, by surface light reflections, or bright light sources such as car headlights, and typically is a consequence of increased forward light scatter within the eye. New bi-xenon high intensity discharge (“HID”) car headlights contain more “blue” light and are often considered as a cause of additional glare disability compared to older headlight sources.

This is of particular importance to macular pigment investigations because of the optical filtration properties of macular pigment. Macular pigment acts as a short wavelength (blue) light filter. Its prereceptoral and central location facilitate the optimization of visual performance with respect to glare because intraocular forward light scatter is short wavelength (blue) light dominated.

Glare disability can be assessed using the Functional Acuity Contrast Test (FACT), as disclosed by Loughman et. al., 2010 Vision Res. 50, 1249-1256.

### (iv) Discomfort Glare

Discomfort glare results in an instinctive desire to look away from a bright light source or difficulty in seeing a task. It refers to the sensation one experiences when the overall illumination is too bright e.g. on a snow field under bright sun.

Macular pigment has the capacity to diminish the effects of discomfort glare because (a) it filters the blue component which contains most energy; less light and less energy therefore reach the photoreceptors to affect performance, and (b) macular pigment also has dichroic properties which means it has the capacity to filter plane polarised light. Plane polarised light is light reflected from a surface (e.g. snow covered ground, water etc) into the eye. It is unidirectional so the energy is concentrated and therefore has increased effect on vision. This is why skiers, anglers and the like wear polarised sunglasses to reduce such discomfort glare. Discomfort glare is assessed using a discomfort rating scale as disclosed by Wenzel et al., 2006 Vision Res. 46, 4615-4622.

### (v) Ocular Straylight

Ocular straylight is a parameter that is relatively new in clinical practice after being studied for many years in experimental settings. It concerns the part of the incident light that is scattered by the ocular media and does not participate in the normal image formation on the retina. Instead, this light creates a more or less homogeneous haze over the retinal image. Several pathologies are known to increase retinal straylight considerably, which may lead to symptoms such as loss of contrast sensitivity, disability glare, and halos. This will reduce a patient's quality of vision in everyday life, for example while driving at night and recognizing a person against a light source, but has only a very limited effect on visual acuity as measured during an ophthalmic examination.

As macular pigment absorbs the dominant short wave scattered component, it has the capacity to significantly reduce the amount of ocular straylight, and therefore further enrich visual performance particularly under circumstances of glare.

Ocular stray light is assessed using the Oculus C-Quant as disclosed by van Bree et al., 2011 Ophthalmology 118, 945-953.

(vi) Photostress Recovery

Photostress Recovery testing is a method of assessing visual performance by timing the recovery of visual function after adaptation to an intense light source. The test involves exposing the macula to a light source bright enough to bleach a significant proportion of the visual pigments. Return of normal retinal function and sensitivity depends on the regeneration of the visual pigments. The test essentially provides an indirect assessment of macular function.

Photostress recovery is assessed using a macular automated photostress test using the Humphrey Perimeter as disclosed by Loughman et. al., 2010 Vision Res. 50, 1249-1256.

(vii) S-cone Sensitivity

S-cones are the “blue” sensitive cones i.e. their peak sensitivity is to short wavelengths. Typically, a person with high levels of macular pigment would be expected to demonstrate low S-cone sensitivity, as the macular pigment is minimising the amount of blue light striking the photoreceptors. Combining a test of S cone sensitivity with a photostress test can provide information on the direct effects of macular pigment on the actual sensitivity of those cones most affected by glare.

S-cone sensitivity is assessed using the short-wavelength automated perimetry program (SWAP) on the Humphrey Perimeter as described by (Davison et. al., Optom. Vis. Sci. 2011 vol. 88).

(viii) Assessment of VP by questionnaire

Another method of testing for improvement in visual performance is the use of a questionnaire to score the subject's own assessment of their visual performance. In preferred embodiments of the invention therefore, a detectable improvement in visual performance is determined by an increased score in a subjective assessment questionnaire following a suitable period of weeks or months of consumption of the composition, as compared to a control assessment questionnaire completed prior to commencing consumption of the composition.

A suitable questionnaire is disclosed by Charalampidou et al., Arch. Ophthalmol. 2011 (May 9^{th}, Epublication ahead of print), in which is described a 30-part, non validated, “Visual Function in Normals” questionnaire (VFNq30), which was designed to assess subjective visual performance improvement. The design was based in part on a previously-validated visual activities questionnaire (Sloane et al., “The Visual Activities Questionnaire: Developing an instrument for assessing problems in everyday visual tasks. Technical Digest, Non-invasive Assessment of the Visual System, Topical Meeting of the Optical Society of America 1992), but adapted to suit a normal, young and healthy population sample. This questionnaire allows the subject to quantify their visual performance using three separate metrics: situational analysis (SA) which requires the subject to rate their visual performance in specified daily life situations; comparative analysis (CA) which requires the subject to compare their perceived visual performance to that of their peers/family/friends; subject satisfaction score (SSS) which requires the subject to provide an overall estimate of their perceived quality of vision. Each of the three metrics above is computed to give a performance score for five different functional aspects of their vision: acuity/spatial vision: glare disability; light/dark adaptation; daily visual tasks; and color discrimination.

Time to achieve an improvement of VP

Obviously, one does not expect any measurable, discernible or detectable improvement in the visual performance of a subject immediately after consuming the composition of the invention. The period of dietary supplementation required to produce a measurable improvement in visual performance will depend on several factors, including the average daily dose size of the macular carotenoids in the subject prior to commencing dietary supplementation, the subject's general health etc. Typically one would expect to require dietary supplementation with the composition of the invention for at least 8 weeks, and more preferably at least 3 or 6 months before measuring one or more visual performance parameters to test for any improvement therein.

The subject may need to consume the active composition of the invention at least once a week, more normally at least 3 times a week, and preferably daily.

An effective amount of the macular pigment-containing composition for a particular subject can readily be determined by non-inventive routine trial and error, in view of the guidance given in the present specification. Low doses can be given initially and the dosage increased until an improvement in visual performance is detected. The subject's visual performance can be tested in any of a number of convenient methods, as elaborated below.

For present purposes, MZ is understood to include within the term esters of MZ, for example the acetate, laurate, myristate, palmitate, linoleate, linolenate and arachidonate esters, and esters with omega 3 fatty acids.

The composition will preferably comprise MZ at a concentration of at least 0.001% w/w up to 20% w/w. In one embodiment, a preferred concentration of MZ may be in the range 3-10% w/w. However, the person skilled in the art will appreciate that the precise concentration of MZ in the composition is not critical: a beneficial effect on the visual performance of the subject can be obtained by consuming larger doses of a composition comprising lower concentrations of MZ and vice versa. A typical effective average daily dose of MZ to be consumed by a normal human adult subject will typically be in the range 0.1 mg to 100 mg per day, more conveniently in the range 1 to 50 mg per day, and preferably in the range 5-25 mg per day.

The composition may conveniently be in unitary dosage form e.g. as a tablet, capsule or the like. Conveniently, but not necessarily, the composition may be packaged in a foil blister pack, of the sort known to those skilled in the art. Desirably one or two of the doses are taken each day, the amount of MZ in the doses being adjusted accordingly.

The composition may desirably comprise not only MZ, but also lutein and/or zeaxanthin. Most preferably the composition will comprise MZ, lutein and zeaxanthin, which may be collectively referred to as macular carotenoids. Conveniently, but not necessarily, MZ will be present in the composition at a greater concentration or the same concentration as lutein or zeaxanthin. The percentage of either MZ or lutein in the composition can range from 10% to 90% (of macular carotenoid pigment present in the formulation). The percentage of zeaxanthin can typically range from 5 to 45% (of macular carotenoid pigment in the formulation). A particularly favoured composition has an MZ: lutein: zeaxanthin ratio of 10:10:2 (or 45%, 45%, 10%).

The three macular carotenoids may be combined or preferably manufactured as such in single formulation. The composition may be in any formulation suitable for oral consumption by a human subject, including a tablet, capsule, gel, liquid, powder or the like. The macular carotenoids may be granulated for example as microcapsules before inclusion in the formulation. The composition may conveniently comprise conventional diluents, especially vegetable oils such as sunflower, safflower, corn oil and rape seed oils, excipients, bulking agents and the like which are well known to those skilled in the art. Such substances include, calcium and/or magnesium stearate, starch or modified starch.

Other conventional formulating agents may be present in the composition, including any one or more of the following non-exclusive list: acidity regulators; anticaking agents (e.g. sodium aluminosilicate, calcium or magnesium carbonate, calcium silicate, sodium or potassium ferrocyanide), antioxidants (e.g. vitamin E, vitamin C, polyphenols), colorings (e.g. artificial colorings such as FD&C Blue No. 1, Blue No. 2, Green No. 3, Red No. 40, Red No. 3, Yellow No. 5 and Yellow No. 6; and natural colorings such as caramel, annatto, cochineal, betanin, turmeric, saffron, paprika etc.); color retention agents; emulsifiers; flavours; flavour enhancers; preservatives; stabilizers; sweeteners and thickeners.

The abovementioned compositions containing MZ can be an added to a preparation containing essential vitamins and minerals; for example a one a day tablet/capsule containing all RDAs of the vitamins and minerals required by man; or dietary products which are fortified by vitamins and minerals; or together with omega 3 fatty acids.

Macular carotenoids containing MZ can be fed to hens and the eggs therefrom can provide an excellent source of MZ for human consumption

Conveniently, the composition may be packaged in unitary dose form e.g. as a plurality of tablets, capsules or pills, which may be packaged loose (e.g. in a tub) or may be packaged individually (e.g. in a blister pack).

Typically the composition will be consumed at least once a week, preferably at least twice a week, more preferably at least three times a week, and most preferably at least daily. In some embodiments the composition may be consumed more than once a day (e.g. once in the morning and once in the evening). The person skilled in the art will appreciate that the frequency of consumption can be adjusted to take account of the concentration of macular pigment carotenoids, especially meso-zeaxantion, present in the formulation.

Consuming the composition over a sufficient period of time (typically at least 8 weeks, preferably at least 3 months, more preferably over at least 6 months, and most preferably for 12 months or more) will typically result in an increase in the level of macular pigment in a subject, and a detectable improvement in at least one measured parameter of visual performance (preferably letter contrast sensitivity).

The amount of increase in the level of macular pigment carotenoids in the subject which is achieved by consumption of the composition may depend on, for example, the level of macular pigment carotenoids present in the subject's eyes prior to commencement of consumption of the composition. Subjects with Alzheimer's disease might be expected to exhibit a statistically significant increase (e.g. p<0.01) in the level of macular pigment following long term (i.e. 3 months or more) consumption of the composition.

Typically the method/composition will produce an improvement of at least 5%, preferably at least 8%, more preferably at least 10%, relative to the same visual performance parameter measured prior to consumption of the composition.

For the avoidance of doubt it is hereby explicitly stated that any feature of the invention described herein as preferable, advantageous, convenient, desirable, typical or the like may be present in any embodiments of the invention in isolation, or in any combination with any one or more other such features, unless the context dictates otherwise. In addition, features described in relation to one aspect of the invention will equally apply to the other aspects of the invention, unless the context dictates otherwise.

The content of all publications and citations mentioned in this specification is specifically incorporated herein by reference.

The invention will now be further described by way of illustrative embodiment and with reference to the accompanying drawings, in which:

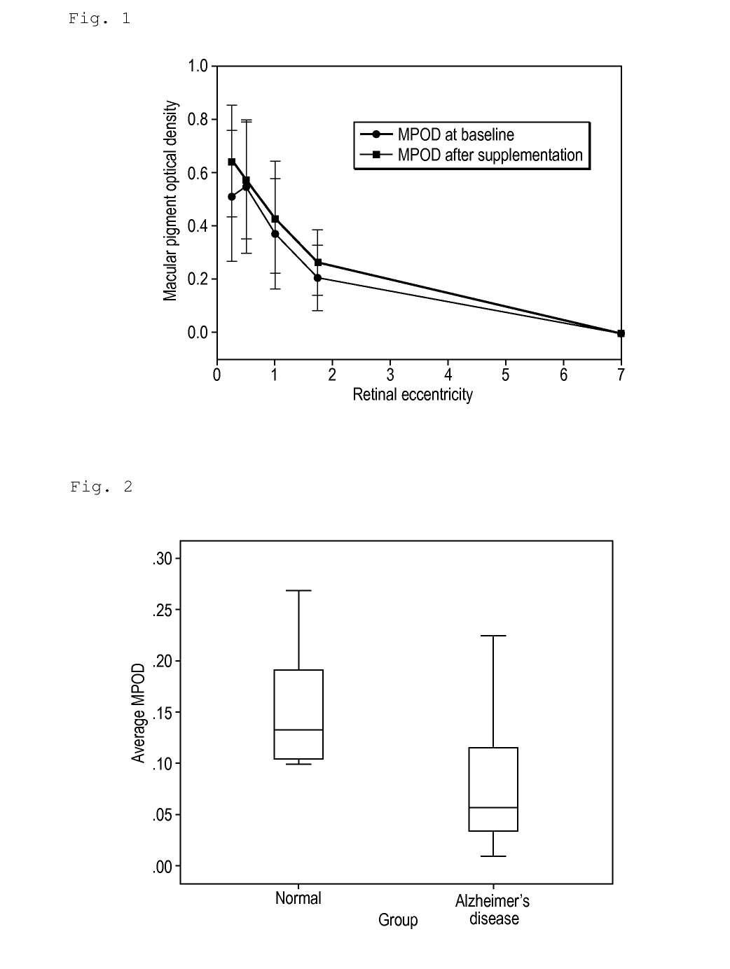

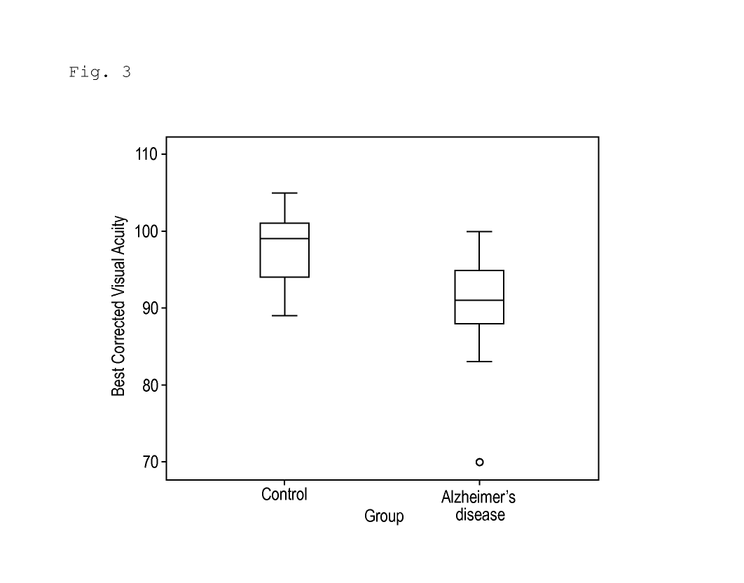

FIG. 1 is a graph of macular pigment optical density (MPOD arbitary units) against retinal eccentricity (degrees), showing the spatial profile of molecular pigment before (at baseline, circular symbols) and after (square symbols) supplementation with a macular pigment composition (containing 10 mg lutein, 10 mg mesozeaxanthin, 2 mg zeaxanthin) in non Alzheimer's subjects with an atypical “central dip” MP profile;

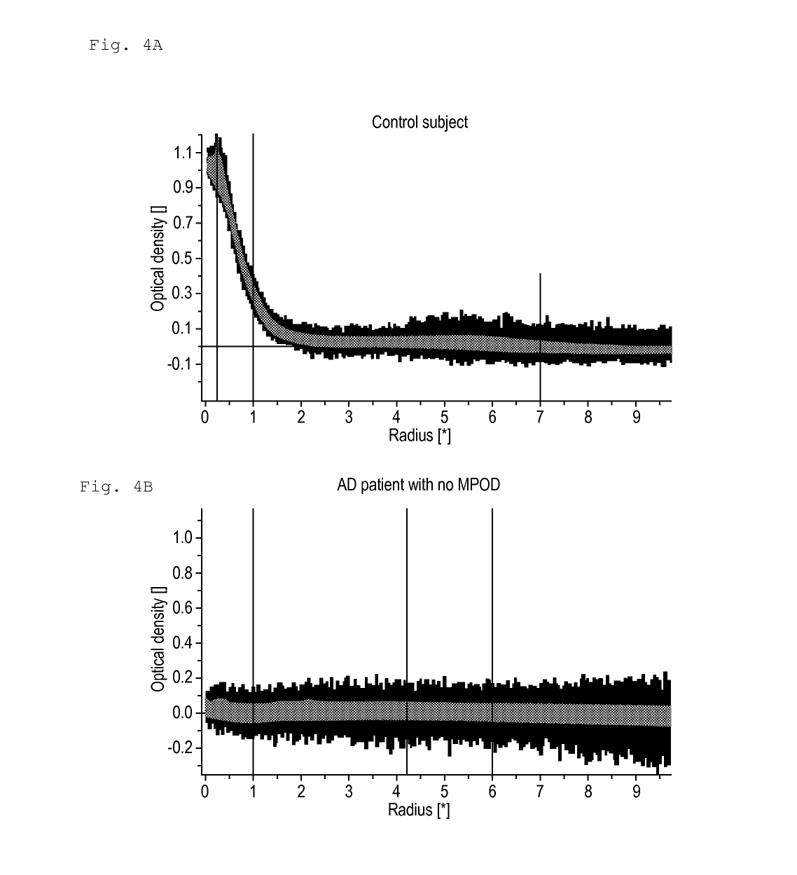

FIG. 2 is a bar chart showing the mean MPOD in normal (control) subjects and subjects with Alzheimer's disease (error bars represent 1.S.D.);

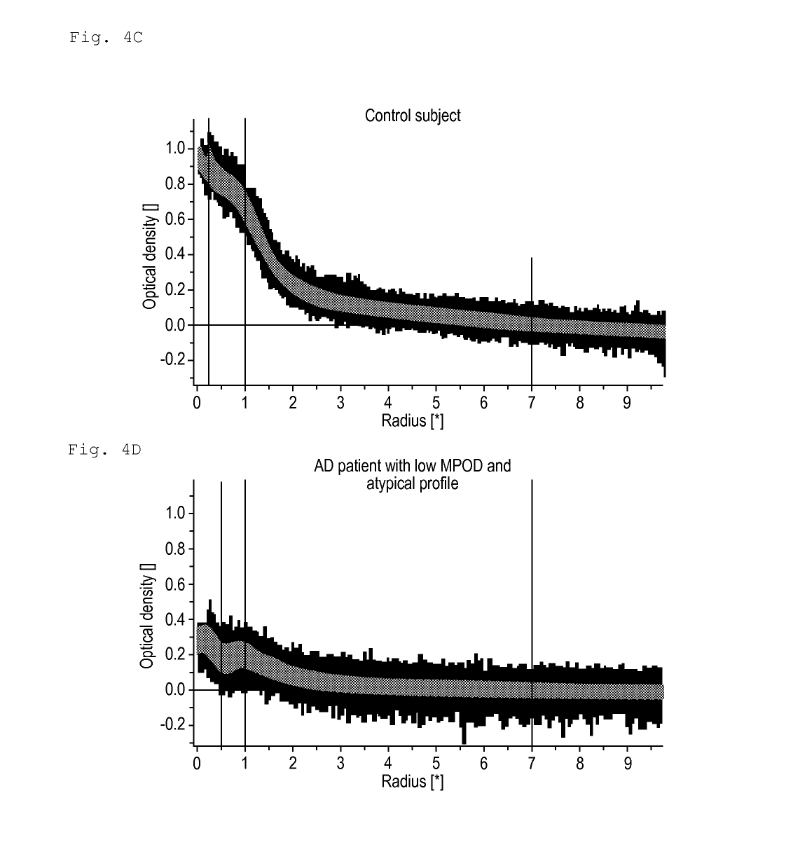

FIG. 3 is a bar chart showing the mean BCVA (best corrected visual ecuity) in normal (control) subjects and subjects with Alzheimer's disease (error bars represent 1.S.D.);

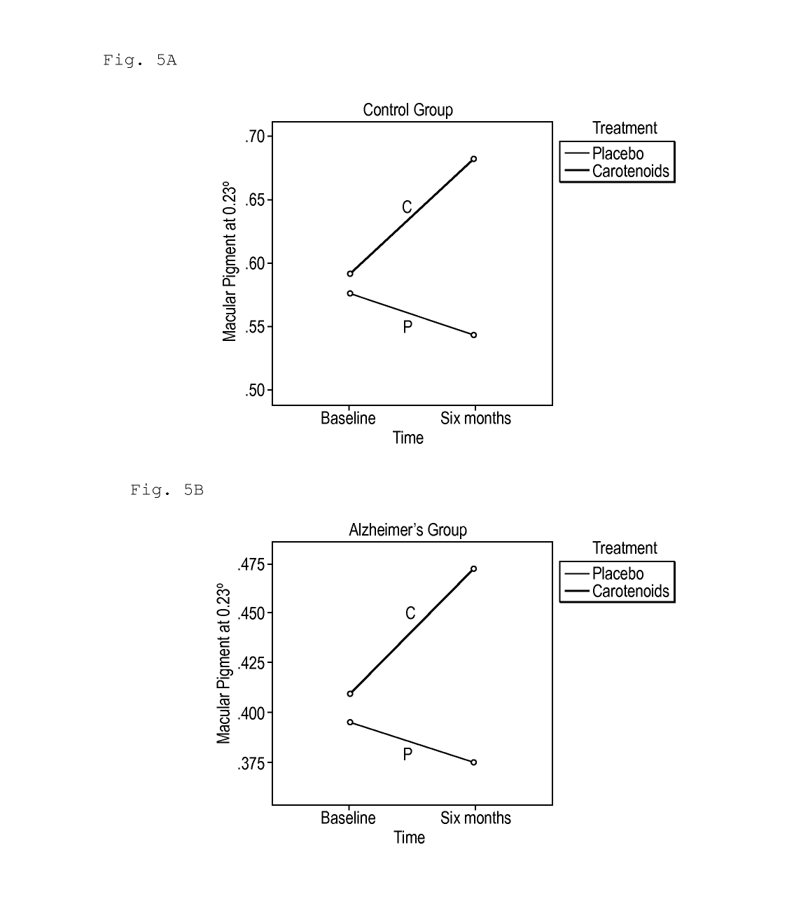

FIG. 4 shows four graphs of MPOD (arbitary units) against radius, illustrating the macular pigment distribution profile for two control subjects (FIG. 4A and FIG. 4C) and two subjects with Alzheimer's disease (FIG. 4B and FIG. 4D). One of the Alzheimer's subjects had no detectable MPOD, and the other had very low levels with an atypical profile;

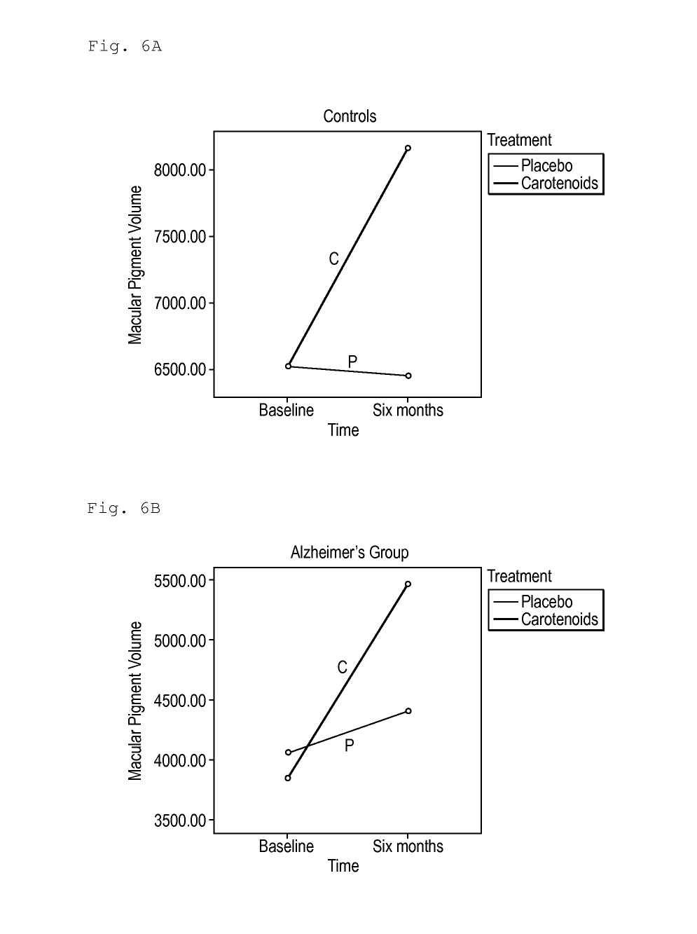

FIG. 5 shows two graphs of macular pigment (MPOD) at 0.23° eccentricity against time for the control group (FIG. 5A) and Alzheimer's Disease group (FIG. 5B), for placebo treatment (p) or carotenoid treatment (c);

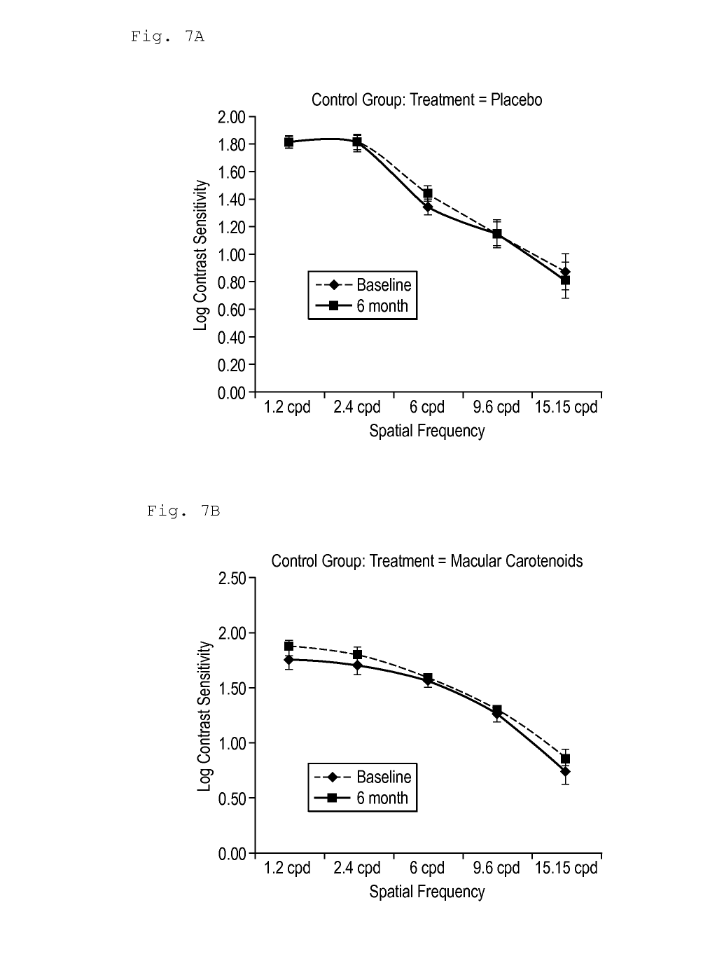

FIG. 6 shows graphs of macular pigment volume (arbitrary units) for control group (FIG. 6A) and Alzheimer's Disease group (FIG. 6B) against time, for placebo treatment (p) or carotenoid treatment (c); and

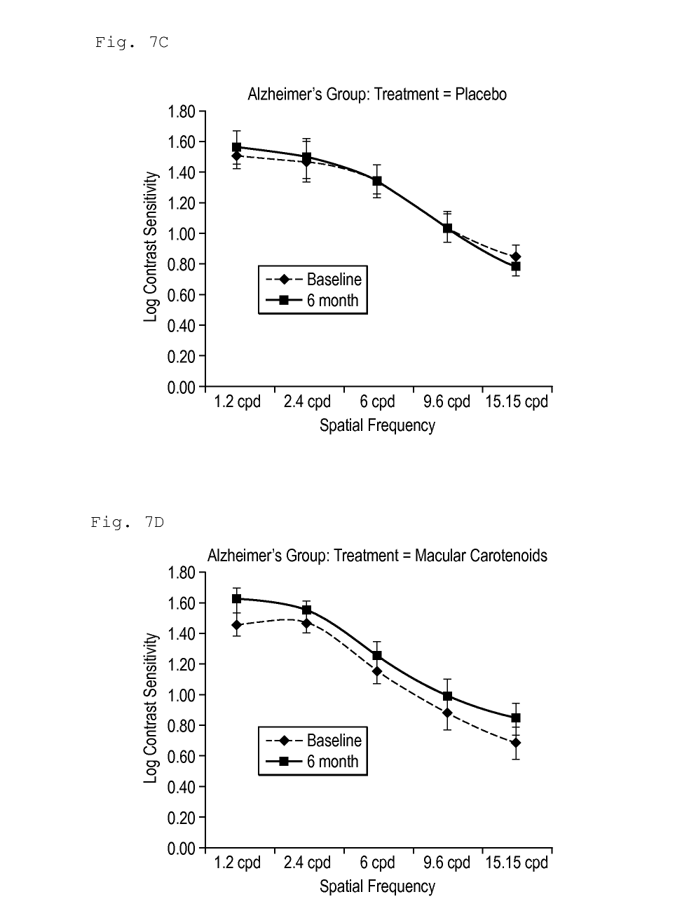

FIG. 7 shows graphs of log contrast sensitivity against spatial frequency for control groups given placebo (FIG. 7A) or carotenoid treatment (FIG. 7B), and Alzheimer's Groups given placebo (FIG. 7C) or carotenoid treatment (FIG. 7D).

### EXAMPLE 1

### Supplementation of a Formulation Containing MZ to Normal People with Atypical Spatial Profiles (“Central Dip”) of their Macular Pigment and the Effect on Visual Performance

Eight subjects with pre-identified atypical MPOD spatial profile (central dips) (Nolan et al., 2012 Experiemtnal Eye Research, 101, 9-15) were recruited into this study. All eight subjects consumed a daily supplement containing 10 mg MZ, 10 mg L, and 10 mg Z for 3 months.

### Methods

MPOD was measured as described by Nolan et al cited above. Contrast sensitivity was measured as described by Loughman et al (2010 Vision Res. 50, 1249-1256).

### Results

1. MPOD results: As seen from Table 3 and FIG. 1, the spatial profile of MP was normalised following supplementation with 10 mg MZ, 10 mg L, and 10 mg Z for 3 months. All subjects responded to this intervention. Statistically significant increases were seen at all eccentricities except for 0.5°.

TABLE 3

| Eccentricity | Baseline | 3 months | P |
|---|---|---|---|
| 0.25° | 0.51 ± 0.25 | 0.64 ± 0.21 | <0.001 |
| 0.5° | 0.54 ± 0.25 | 0.57 ± 0.20 | 0.140 |
| 1° | 0.37 ± 0.20 | 0.43 ± 0.21 | 0.016 |
| 1.75° | 0.20 ± 0.12 | 0.26 ± 0.12 | 0.008 |

2. Contrast sensitivity: As seen from Table 4 there was also an improvement in contrast sensitivity following supplementation with 10 mg MZ, 10 mg L, and 10 mg Z for 3 months.

TABLE 4

| Contrast | | | |
|---|---|---|---|
| sensitivity | Baseline | 3 months | p |
| 1.2 cpd | 2.00 ± 0.15 | 2.07 ± 0.12 | 0.103 |
| 2.4 cpd | 1.86 ± 0.16 | 2.02 ± 0.19 | 0.003 |
| 6 cpd | 1.56 ± 0.19 | 1.71 ± 0.21 | <0.001 |
| 9.6 cpd | 1.34 ± 0.21 | 1.46 ± 0.18 | 0.051 |
| 15.15 cpd | 1.02 ± 0.16 | 1.11 ± 0.20 | 0.035 |

3. The relationship between change in MPOD 0.25 and the change in letter contrast sensitivity (CS) at each spatial frequency is presented in Table 5 below. A strong positive relationship is observed between increase in central MP and improvement in contrast sensitivity. This finding suggests that the rebuilding of “central dips” i.e. the increase in central MPOD, has important implications for vision, as measured by letter CS, and uniquely in subjects presenting with central dips at baseline.

TABLE 5

| | Change | | Change | | Change | | Change | | Change | |
|---|---|---|---|---|---|---|---|---|---|---|
| | in CS | | in CS | | in CS | | in CS | | in CS | |
| | 1.2cpd | | 2.4cpd | | 6cpd | | 9.6cpd | | 14.15cpd | |
| Change in MPOD | r | p | R | p | r | p | r | p | r | p |
| 0.25 | 0.803 | 0.017 | 0.831 | .011 | 0.610 | .108 | .677 | 0.065 | .280 | 0.503 |

### Conclusions

In Ireland about 12% of normal people have atypical (central dip) profiles of macular pigment (Kirby et al 2010 Opthalmol. Vision Sci. 51 6722-6728.) This example shows that the administration of a formulation containing macular carotenoids will result in an improvement in visual performance (as measured by letter contrast sensitivity), in subjects with an atypical macular pigment profile.

### EXAMPLE 2

### Introduction

The object of this study was to compare MPOD, VP and Cognition of Alzheimer patients (AD) with those of age-matched controls. It was conducted by the Macular Pigment research Group at the Waterford Institute of Technology, Waterford, Republic of Ireland. Ethical permission was obtained.

### Methods

### Recruitment of Subjects and Obtaining Informed Consent

AD is predominantly a clinical diagnosis supported by neurophysiological testing. Subjects who meet the inclusion criteria (i.e. patients diagnosed with moderate AD, who have demonstrated capacity to consent) were identified and recruited directly from the local Waterford Regional Hospital. We recruited 14 patients with moderately severe AD and 14 age-matched controls for this study. Total number of subjects=28.

Lifestyle information: lifestyle factors (e.g. tobacco use) were recorded by questionnaire.

Health information: blood pressure levels and body mass index were also recorded for each subject.

Dietary questionnaire: a food questionnaire designed to estimate carotenoid intake was used to estimate the intake of L and Z in the diet.

Cognitive function: Cognitive function was assessed using the standard MMSE (mini-mental state examination), FAS (phonemic fluency score) and Animal fluency score (semantic fluency score). Cognitive function was also measured and assessed using a technology platform called CANTAB® (Cambridge Neuropsychological Test Automated Battery [www.cambridgecognition.com]), which allowed for investigation of cognitive function and its relationship with MP in patients with and without AD.(ref. 1)

Macular pigment: MP was measured using the dual-wavelength fundus autofluorescence (AF) technique using the newly available Spectralis Heidelberg Engineering technology(ref. 2). The AF technique is based on detection of autofluorescence derived from lipofuscin at two specific wavelengths (488 nm and 514 nm), and is therefore less susceptible to reflectance artefacts which present challenges for other methods. As absorption of MP at 488 nm is high, and at 514 nm is close to zero, total MP and its topographic map can be determined It is important to note that the 2-wavelength AF-method provides a single-pass measurement of the MP with no assumptions about the pathway through the pigment. Recent developments using AF have enabled topographic maps of MP to be generated.

Retinal photograph: A retinal photograph was also taken from each patient to assess the health of the retina and check for presence of ocular pathology. These photographs were reviewed by a Consultant Ophthalmologist (Whitfield Clinic, Waterford, Ireland). Patients with Glaucoma were not recruited into the study for safety reasons given the need to dilate the pupil.

Visual performance:

Best Corrected Visual Acuity

BCVA was measured using computerised logMAR ETDRS test chart (Test Chart 2000 Xpert; Thomson Solutions) viewed at 3 meters. The Sloane early Diabetic Treatment Retinography Study (ETDRS) letter set was used for the test. At the first incompletely read line, the letters of the line were randomised three times using the testing software's randomised function and an average of three scores taken. The BCVA was recorded in visual acuity rating (VAR).

### Contrast Sensitivity

Letter CS was assessed using the computerised LogMAR RTDS test chart (2000 PRD) at five spatial frequencies (1.2, 2.4, 6.0, 9.6, 15.15 cpd). The Sloan optotypes were chosen and the subject asked to read the letters aloud while fixating on the chart at a distance of 6 m. The letter set was randomised during each change. The percentage contrast of letter optotypes was decreased in 0.25 log CS steps until the lowest contrast value for which the subject could see at least three letters was reached. The test was then repeated for the other spatial frequencies. Each letter had a nominal log CS value of 0.03. Missed letters at any contrast level were noted. The resultant log CS value for the subject at a particular spatial frequency was calculated by adding any extra letters and/or subtracting missed letters from best log CS value corresponding to the lowest percentage contrast.

### Statistics

The statistical package IBM SPSS version 21 was used for all statistical analyses. Inter-group differences at baseline (e.g. AD versus controls) were analysed using independent samples t-tests or chi-squared tests as appropriate. The 5% level of significance was used throughout the analysis, without adjustment for multiple comparisons.

Results:

Relationship between MPOD and AD

Data was available for 32 eccentricities from 0 degrees of eccentricity out as far as 9 degrees of eccentricity. MPOD was statistically significantly lower for patients with AD when compared the control subjects (see below Table 6).

TABLE 6
MPOD in Alzheimer Disease patients and Controls
Group Statistics

| | | | | Std. | P |
|---|---|---|---|---|---|
| Group | | N | Mean | Deviation | value |
| AFMP 0 | Alzheimer's disease | 14 | .4207 | .27971 | .010 |
| | Normal | 14 | .6836 | .21525 | |
| AFMP 0.23 | Alzheimer's disease | 14 | .3957 | .23566 | .015 |
| | Normal | 14 | .6129 | .20284 | |
| AFMP 0.51 | Alzheimer's disease | 14 | .3114 | .21817 | .008 |
| | Normal | 14 | .5293 | .18424 | |
| AFMP 0.74 | Alzheimer's disease | 14 | .2750 | .20979 | .003 |
| | Normal | 14 | .5093 | .16546 | |
| AFMP 1.02 | Alzheimer's disease | 14 | .2250 | .17253 | .002 |
| | Normal | 14 | .4450 | .17226 | |
| AFMP 1.25 | Alzheimer's disease | 14 | .1700 | .13416 | .002 |
| | Normal | 14 | .3614 | .16176 | |
| AFMP 1.52 | Alzheimer's disease | 14 | .1250 | .09835 | .005 |
| | Normal | 14 | .2693 | .14531 | |
| AFMP 1.76 | Alzheimer's disease | 14 | .0943 | .08045 | .006 |
| | Normal | 14 | .2114 | .12165 | |
| AFMP 1.99 | Alzheimer's disease | 14 | .0750 | .06992 | .006 |
| | Normal | 14 | .1686 | .09526 | |
| AFMP 2.23 | Alzheimer's disease | 14 | .0607 | .06281 | .008 |
| | Normal | 14 | .1371 | .07740 | |
| AFMP 2.5 | Alzheimer's disease | 14 | .0500 | .05477 | .006 |
| | Normal | 14 | .1171 | .06318 | |
| AFMP 2.77 | Alzheimer's disease | 14 | .0421 | .04886 | .005 |
| | Normal | 14 | .1029 | .05608 | |
| AFMP 3.01 | Alzheimer's disease | 14 | .0393 | .04305 | .005 |
| | Normal | 14 | .0914 | .04737 | |
| AFMP 3.24 | Alzheimer's disease | 14 | .0350 | .03674 | .003 |
| | Normal | 14 | .0829 | .04027 | |
| AFMP 3.52 | Alzheimer's disease | 14 | .0307 | .03149 | .002 |
| | Normal | 14 | .0757 | .03589 | |
| AFMP 3.75 | Alzheimer's disease | 14 | .0264 | .03296 | .002 |
| | Normal | 14 | .0686 | .03207 | |
| AFMP 4.02 | Alzheimer's disease | 14 | .0236 | .02845 | .001 |
| | Normal | 14 | .0636 | .02845 | |
| AFMP 4.26 | Alzheimer's disease | 14 | .0221 | .02547 | .001 |
| | Normal | 14 | .0564 | .02373 | |
| AFMP 4.49 | Alzheimer's disease | 14 | .0200 | .02287 | .001 |
| | Normal | 14 | .0521 | .02190 | |
| AFMP 4.77 | Alzheimer's disease | 14 | .0179 | .01929 | .000 |
| | Normal | 14 | .0479 | .02007 | |
| AFMP 5 | Alzheimer's disease | 14 | .0136 | .01598 | .000 |
| | Normal | 14 | .0421 | .01578 | |
| AFMP 5.27 | Alzheimer's disease | 14 | .0136 | .01499 | .000 |
| | Normal | 14 | .0371 | .01590 | |
| AFMP 5.51 | Alzheimer's disease | 14 | .0114 | .01027 | .000 |
| | Normal | 14 | .0293 | .01141 | |
| AFMP 5.74 | Alzheimer's disease | 14 | .0107 | .00997 | .000 |
| | Normal | 14 | .0271 | .00825 | |
| AFMP 6.02 | Alzheimer's disease | 14 | .0079 | .00893 | .001 |
| | Normal | 14 | .0207 | .00917 | |
| AFMP 6.25 | Alzheimer's disease | 14 | .0050 | .00760 | .000 |
| | Normal | 14 | .0171 | .00825 | |
| AFMP 6.52 | Alzheimer's disease | 14 | .0036 | .00497 | .000 |
| | Normal | 14 | .0136 | .00497 | |
| AFMP 6. 76 | Alzheimer's disease | 14 | .0021 | .00579 | .003 |
| | Normal | 14 | .0100 | .00679 | |
| AFMP 6.99 | Alzheimer's disease | 14 | −.0014 | .00535 | .008 |
| | Normal | 14 | .0043 | .00514 | |

The average MPOD across its spatial profile was then calculated for each subject. This average value was obtained from the 32 eccentricity values provided by the Spectralis software. The average MPOD in patients with AD was also statistically significant lower when compared to the control subjects (FIG. 2). Given the small sample size of this study, this is a surprisingly clear and dramatic result.

The AD patients were comparable to the control subjects in all variables including age, sex, BMI and diet.

Comparing cognitive function measures for patients with and without Alzheimer's disease

Table 7 presents the difference in cognitive function parameters between patients with Alzheimer's disease and normal (control) subjects. As expected, all cognitive function outcomes were statistically significantly inferior for patients with AD when compared to the normal (control) subjects.

TABLE 7
Comparing cognitive function measures for patients
with and without Alzheimer's disease

| | | | Std. | P |
|---|---|---|---|---|
| Test | Group | Mean | Deviation | value |
| MMSE¹ | Alzheimer's disease | 18.83 | 2.823 | 0.000 |
| | Normal | 29.50 | 1.345 | |
| FAS score² | Alzheimer's disease | 19.38 | 11.856 | 0.014 |
| | Normal | 33.54 | 11.494 | |
| Animal fluency | Alzheimer's disease | 6.50 | 3.024 | 0.000 |
| score³ | Normal | 15.38 | 3.305 | |
| VRM⁴ immediate free | Alzheimer's disease | 1.67 | .516 | 0.000 |
| recall correct | Normal | 6.89 | 1.691 | |
| PAL⁵ total errors | Alzheimer's disease | 132.67 | 7.474 | 0.000 |
| (lower score is | Normal | 56.44 | 35.444 | |
| better) | | | | |
| VRM⁴ delayed | Alzheimer's disease | 16.80 | 3.701 | 0.000 |
| recognition correct | Normal | 23.78 | .441 | |

¹Mini-Mental State Examination (used to screen for cognitive impairment in clinical practice)
²Phonemic fluency score (F, A and S are the letters used in it)
³Semantic fluency score
⁴Verbal Recognition Memory test, assesses immediate and delayed memory of verbal information under free recall and forced choice recognition
⁵Paired Associates Learning task, assesses visual memory

Relationship between measures of cognitive function and MP

We found that there is a strong positive correlation between average MPOD and a large number of cognitive indices (see Table 8 [Linear Regression] and e.g. FIG. 2).

TABLE 8
The relationship between measures of cognitive function and
average MPOD for all subjects (patients with and without AD)

| | Correlation with | |
|---|---|---|
| Measures of | Average MPOD | |
| cognitive function | R-value | p-value |
| MMSE¹ | 0.516 | 0.007 |
| FAS score² | 0.777 | 0.000 |
| Animal fluency score³ | 0.646 | 0.002 |
| VRM⁴ immediate free recall | 0.697 | 0.004 |
| correct | | |
| VRM⁴ immediate recognition | 0.682 | 0.010 |
| correct | | |
| PAL⁵ total errors | 0.715 | 0.003 |
| VRM⁴ delayed recognition | 0.711 | 0.004 |
| correct | | |

¹Mini-Mental State Examination (used to screen for cognitive impairment in clinical practice)
²Phonemic fluency score (F, A and S are the letters used in it)
³Semantic fluency score
⁴Verbal Recognition Memory test, assesses immediate and delayed memory of verbal information under free recall and forced choice recognition.
⁵Paired Associates Learning task, assesses visual memory and learning.

Comparing Visual Performance Measures for Patients with and without Alzheimer's Disease

Table 9 presents the mean±SD values of visual performance parameters for patients with Alzheimer's disease and normal (control) subjects.

BCVA and CS at 9.0 cpd were statistically significantly inferior for patients with AD when compared to the normal (control) subjects (FIG. 3). All other visual performance outcomes were lower in patients with AD but not statistically significant.

TABLE 9
Visual performance in Alzheimer Disease and Control subjects

| Test | Group | Mean | Std. Deviation | P value |
|---|---|---|---|---|
| BCVA | Alzheimer's disease | 90.85 | 8.305 | 0.019 |
| | Normal | 97.50 | 5.244 | |
| CS 1.2 cpd | Alzheimer's disease | 1.6140 | .25290 | 0.242 |
| | Normal | 1.8017 | .23859 | |
| CS 2.4 cpd | Alzheimer's disease | 1.5291 | .21002 | 0.090 |
| | Normal | 1.7825 | .19335 | |
| CS 6.0 cpd | Alzheimer's disease | 1.2645 | .27948 | 0.108 |
| | Normal | 1.4417 | .22639 | |
| CS 9.0 cpd | Alzheimer's disease | 0.9191 | .37596 | 0.007 |
| | Normal | 1.1633 | .28089 | |
| CS 15.15 cpd | Alzheimer's disease | 0.5945 | .40712 | 0.089 |
| | Normal | 0.7842 | .34757 | |

BCVA = best corrected visual acuity; CS = contrast sensitivity; cpd = cycles per degree

### Relationship Between Measures of Visual Performance and MP

### Corrected Visual Acuity

Analysing the group as a whole (AD patients and control subjects), there is no statistically significant relationship between average MPOD and corrected visual acuity (r=0.236, p=0.236). Moreover, the relationship between average MPOD and corrected visual acuity remained non-significant when the subjects were split into normal and AD groups (r=0.082, p=0.779 and 0.035, p=0.910, respectively).

### Contrast Sensitivity

Analysing the group as a whole (AD patients and control subjects), there is no statistically significant relationship between average MPOD and contrast sensitivity at any spatial frequency (r=0.083 to r=0.232). Moreover, the relationship between average MPOD and contrast sensitivity remained non-significant when the subjects were split into normal and AD groups (r=0.81 to 0.469).

Relationship Between AD and Atypical Profiles This study, for the first time, assessed MP profile in patients with AD. 7 out of 14 AD subjects (50%) displayed atypically low MP centrally compared to only 1 out of 14 (7%) in the control group (chi-square: p=0.012). In addition one patient had no macular pigment. In the general population the prevalence of the atypical profile is in circa 12% of individuals. ((3)) Our sample percentage of 50% for AD subjects is significantly different from the normal population of 12% prevalence of an atypical profile (p<0.001).

### Prevalence of AMD in Study

Of interest, 27% of the patients with AD also had confirmed presence of age-related macular degeneration (confirmed following an ophthalmic examination), compared to 15% of patients in the control group who were confirmed of having age-related macular degeneration (chi-square; p=0.475).

Conclusions

Alzheimer Disease patients have a much lower MPOD and exhibit a much greater incidence of atypical (central dip) profiles than age-matched controls or that occurring in the normal population.

### EXAMPLE 3

### Introduction

This study was conducted to investigate the effect of supplementation with the three macular carotenoids (meso-zeaxanthin [MZ], lutein [L] and zeaxanthin [Z]) in patients with Alzheimer's disease (AD) and controls with respect to the following outcome measures: macular pigment optical density (MPOD); vision performance; and cognitive function. It was conducted by the Macular Pigment Research Group at the Waterford Institute of Technology, Waterford, Republic of Ireland. Ethical permission was obtained.

### Methods

Design:

30 Patients with moderate AD were recruited into the study and in a 50/50 double-blind, placebo-controlled fashion and were treated with either a supplement containing 10 mg MZ; 10 mg L; 2 mg Z (the Carotenoids) or a Placebo. An equal number (n=30) of age-matched controls free of AD were also recruited and again in a 50/50 double-blind, placebo-controlled fashion supplemented with either the supplement or Placebo. The code was broken at 6 months.

### Measurements

MPOD, Best Corrected Visual Acuity (BCVA), Contrast sensitivity (CS), Cognition and Diet assessments were measured by techniques described in Example 2. The statistical package IBM SPSS version 21 was used for all statistical analyses.

### Results

Baseline:

Table 10 presents baseline statistics for outcome variables. These differed significantly between AD and control groups. Of interest, AD subjects had lower MPOD, poorer vision (BCVA and CS) and cognitive function (e.g. FAS) and diet score. Although an attempt was made to match the AD and control groups on age, it can be seen that the AD group is older on average. For this reason further analysis was controlled for age and also for diet score.

TABLE 10
Table 1. Group Statistics at Baseline

| Variable Group | | Mean | Stdev | Sig |
|---|---|---|---|---|
| MPOD at 0.23° | Control | 0.56 | 0.17 | 0.002 |
| | AD | 0.41 | 0.21 | |
| BCVA | Control | 96 | 8.55 | 0.009 |
| | AD | 89 | 11.38 | |
| CS120 | Control | 1.75 | 0.22 | 0.000 |
| | AD | 1.49 | 0.23 | |
| CS15 | Control | 1.18 | 0.26 | 0.005 |
| | AD | 0.95 | 0.30 | |
| CS24 | Control | 1.42 | 0.25 | 0.004 |
| | AD | 1.20 | 0.31 | |
| CS60 | Control | 1.75 | 0.26 | 0.000 |
| | AD | 1.48 | 0.29 | |
| FAS | Control | 33 | 13.84 | 0.000 |
| | AD | 16 | 10.26 | |
| Age (years) | Control | 76 | 6.29 | 0.031 |
| | AD | 80 | 7.81 | |
| Dietary L and Z | Control | 24 | 13.62 | 0.01 |
| | AD | 16 | 8.02 | |

Change in Variables after Six Months of Supplementation

### 1. MPOD at 0.23° Eccentricity

As seen in Table 11, treatment with the macular carotenoids significantly increased MPOD at 0.23° (i.e. central macular pigment) at six months compared to the placebo (p<0.001 from the repeated measures analysis). Moreover, the repeated measures analysis showed no difference in the effect of supplementation between AD subjects and controls (p=0.387, non-significant, for this interaction effect). Also, paired t test assessment (represented by Sig in Table 11) shows the statistical significance of the improvement in MPOD. These findings are illustrated in FIG. 5. The graphs in FIG. 5 show the change in central (0.23° eccentricity) macular pigment optical desity (MPOD) for both the control and AD groups at six months. As seen in this figure, there was a significant improvement in MPOD in both groups when the treatment was Carotenoids. The placebo treatment demonstrated no significant change in MPOD.

TABLE 11

| | | | | Mean | | |
|---|---|---|---|---|---|---|
| | | | | MP at | | |
| | | Mean MP | | 0.23° at | | |
| | | at 0.23° | St | six | St | |
| Group | Treatment | baseline | dev | months | dev | Sig. |
| Control | Placebo | 0.58 | 0.18 | 0.54 | 0.18 | 0.30 |
| | Carotenoids | 0.58 | 0.18 | 0.68 | 0.19 | 0.002 |
| AD | Placebo | 0.40 | 0.17 | 0.38 | 0.16 | 0.089 |
| | Carotenoids | 0.41 | 0.26 | 0.48 | 0.26 | 0.039 |
| AD and | Placebo | 0.50 | 0.19 | 0.47 | 0.19 | 0.10 |
| Controls | Carotenoids | 0.50 | 0.23 | 0.58 | 0.24 | <0.001 |

2. Macular Pigment Volume As seen in Table 12, supplementation with the macular carotenoids significantly increases MP volume (i.e. total MPOD across the spatial profile) at six months compared to the placebo (p<0.001 from the repeated measures analysis). Moreover, the repeated measures analysis showed no difference in the effect of supplementation between AD subjects and controls (p=0.476, non-significant, for this interaction effect). Also, paired t test assessment (represented by Sig in Table 12) shows the statistical significance of the improvement in MPOD. These findings are illustrated in FIG. 6.

FIG. 6 presents the change in the volume of macular pigment optical density (MPOD) across its spatial profile, for both the control and AD groups, at six months. As seen in this figure, there was a significant improvement in MPOD in both groups when the treatment was Carotenoids. The placebo treatment demonstrated no significant change in MPOD.

TABLE 12

| | | Mean MP | | Mean MP | | |
|---|---|---|---|---|---|---|
| | | volume | St | volume at | St | |
| Group | Treatment | baseline | dev | six months | dev | Sig. |
| Control | Placebo | 6543 | 2150 | 6473 | 2132 | 0.394 |
| | Carotenoids | 6593 | 2117 | 8291 | 2692 | <0.001 |
| AD | Placebo | 4009 | 2084 | 4328 | 1948 | 0.34 |
| | Carotenoids | 3805 | 2255 | 5408 | 3130 | 0.001 |
| AD and | Placebo | 5511 | 2439 | 5599 | 2288 | 0.273 |
| Controls | Carotenoids | 5255 | 2568 | 6907 | 3206 | <0.001 |

### 3. Visual Performance

### 3.1 Visual Acuity

There was no significant change in BCVA by six months for either the AD or Control group for any treatment (p>0.05, for all).

### 3.2 Contrast Sensitivity

As seen in Table 13, supplementation with macular carotenoids significantly increased contrast sensitivity at 1.20 cpd at six months compared to the placebo (p<0.039 from the repeated measures analysis). Moreover, paired t test assessment (represented by Sig in Table 13) shows the improvement in CS at many other spatial frequencies. These findings are also illustrated in FIG. 7 which shows an improvement in contrast sensitivity in control and AD subjects. FIG. 7 presents the change in contrast sensitivity for each spatial frequency tested for both the control and AD groups. As seen in this figure, there was an improvement in contrast sensitivity at some spatial frequencies in the control group when the treatment was Carotenoids, and there was improvements at all contrast sensitivity spatial frequencies in the AD group when the treatment was Carotenoids. The placebo treatment demonstrated no significant change in contrast sensitivity for either group. Of note, the greatest improvement in CS was seen in the AD group.

TABLE 13

| | | CS at | | | | |
|---|---|---|---|---|---|---|
| | | 1.2 CPD | | CS at 1.2 CPD | | |
| Group | Treatment | baseline | Stdev | six months | Stdev | Sig. |
| Control | Placebo | 1.83 | 0.15 | 1.82 | 0.15 | 0.84 |
| | Carotenoids | 1.76 | 0.27 | 1.87 | 0.26 | 0.006 |
| AD | Placebo | 1.51 | 0.27 | 1.56 | 0.32 | 0.108 |
| | Carotenoids | 1.47 | 0.25 | 1.63 | 0.24 | 0.040 |
| | | CS at | | | | |
| | | 2.4 CPD | | CS at 2.4 CPD | | |
| Group | Treatment | baseline | Stdev | six months | Stdev | Sig. |
| Control | Placebo | 1.81 | 0.18 | 1.82 | 0.18 | 0.600 |
| | Carotenoids | 1.73 | 0.35 | 1.81 | 0.29 | 0.038 |
| AD | Placebo | 1.47 | 0.41 | 1.48 | 0.40 | 0.461 |
| | Carotenoids | 1.48 | 0.23 | 1.55 | 0.24 | 0.048 |
| | | CS at | | | | |
| | | 6 CPD | | CS at 6 CPD | | |
| Group | Treatment | baseline | Stdev | six months | Stdev | Sig. |
| Control | Placebo | 1.38 | 0.24 | 1.46 | 0.21 | 0.275 |
| | Carotenoids | 1.57 | 0.20 | 1.59 | 0.16 | 0.685 |
| AD | Placebo | 1.35 | 0.26 | 1.34 | 0.31 | 0.785 |
| | Carotenoids | 1.17 | 0.26 | 1.26 | 0.30 | 0.16 |
| | | CS at | | | | |
| | | 9.6 CPD | | CS at 9.6 CPD | | |
| Group | Treatment | baseline | Stdev | six months | Stdev | Sig. |
| Control | Placebo | 1.16 | 0.29 | 1.17 | 0.35 | 0.919 |
| | Carotenoids | 1.27 | 0.22 | 1.32 | 0.17 | 0.380 |
| AD | Placebo | 1.03 | 0.27 | 1.04 | 0.28 | 0.840 |
| | Carotenoids | 0.88 | 0.30 | 0.99 | 0.33 | 0.011 |
| | | CS | | | | |
| | | at 15.15 | | CS at 15.15 | | |
| | | CPD | | CPD six | | |
| Group | Treatment | baseline | Stdev | months | Stdev | Sig. |
| Control | Placebo | 0.89 | 0.31 | 0.83 | 0.32 | 0.39 |
| | Carotenoids | 0.75 | 0.36 | 0.88 | 0.25 | 0.176 |
| AD | Placebo | 0.85 | 0.16 | 0.79 | 0.16 | 0.471 |
| | Carotenoids | 0.68 | 0.24 | 0.85 | 0.24 | 0.047 |

### 4. Cognitive Function

Supplementation with macular carotenoids did not significantly improve any of the cognitive function scores by six months in either the AD or control groups.

### 5. Prevalence of Age-Related Macular Degeneration (AMD) in Study Groups

Of interest, 48% of the patients with AD also had confirmed presence of AMD (confirmed following an ophthalmic examination by Professor Stephen Beatty), compared to only 16% of patients in the control group who were confirmed of having AMD (chi-square; p=0.007).

### Conclusions

This study reports the following:—

MPOD is lower in patients with AD when compared to control subjects

Subjects with AD have a significantly higher prevalence of AMD compared to controls

Treatment with the macular carotenoids (MZ, L, and Z) in AD and control subjects had the following effects after six months supplementation

(a) increased MPOD

(b) increased MP volume across the spatial profile

(c) increased visual performance, as measured by contrast sensitivity

(d) no effect on cognitive function

### REFERENCES

(1) Egerhazi A, Berecz R, Bartok E, Degrell I. Automated Neuropsychological Test Battery (CANTAB) in mild cognitive impairment and in Alzheimer's disease. Prog. Neuropsychopharmacol. Biol. Psychiatry 2007; 31(3):746-751.
(2) Dietzel M, Zeimer M, Heimes B, Pauleikhoff D, Hense H W. The ringlike structure of macular pigment in age-related maculopathy: results from the Muenster Aging and Retina Study (MARS). Invest. Ophthalmol. Vis. Sci. 2011; 52(11):8016-8024.
(3) Kirby M L, Bearrt S, Loane E, Akkali M, Connoly E, Stack J et al. A central Dip in the Macular Pigment Spatial Profile is associated with Age and Smoking. Opthalmol. Vis. Sci. 2010:51(12):6722-6728

1. A method of identifying a human subject, more likely than a subject selected at random from the general population, to have one or more of the following: (i) a low macular pigment concentration in the eye or eyes; (ii) a low visual performance, or (iii) an atypical “central dip” macular pigment distribution; the method comprising the steps of: measuring at least one cognitive function of the subject; comparing the measured cognitive function with a pre-determined threshold; and, if the measured cognitive function is below the threshold, declaring the subject as being more likely to have one or more of low macular pigment concentration in the eye or eyes, low visual performance or an atypical ‘central dip’ macular pigment profile.

2. A method according to claim 1, wherein the subject is suspected, or diagnosed, as having Alzheimer's disease.

3. A method according to claim 1, further comprising the step of administering an effective amount of a macular pigment-containing composition to the subject.

4. A method according to claim 3, wherein the composition comprises mesozeaxanthin.

5. A method according to claim 3, performance of which increases the macular pigment concentration in the eye or eyes of the subject.

6. A method according to claim 3, performance of which produces a detectable improvement in at least one parameter of visual performance in the subject.

7. A method according to claim 6, wherein the parameter is letter contrast sensitivity.

8. A method of improving the visual performance of a human subject without the need to test the macular pigment concentration in the eye or eyes of the subject, the method comprising the steps of: identifying a subject likely to have one or more of (i) low macular pigment concentration; (ii) low visual performance or (iii) an atypical ‘central dip’ macular pigment distribution in accordance with claim 1; and administering an amount of a macular pigment-containing composition sufficient to improve the visual performance of the subject.

9. Use of a macular pigment-containing composition to improve the visual performance of a subject suffering from Alzheimer's disease.

10. A use according to claim 9, wherein the macular pigment-containing composition comprises mesozeaxanthin.

11. A use according to claim 9, wherein the macular pigment-containing composition comprises lutein and/or zeaxanthin.

12. A use according to claim 9, wherein the subject is identified by performing the method of claim 1.
